(19) United States
(12) Patent Application Publication
(10) Pub. No.: US 2026/0182899 A1
(43) Pub. Date: Jul. 2, 2026

(54) Implantable Medical Device Configured to Compute a Burden Measure

(71) Applicant: BIOTRONIK SE & CO. KG, Berlin (DE)

(72) Inventors: Joseph Theodore MARMERSTEIN, Hudson, OH (US); Ravi Kiran Kondama REDDY, Portland, OR (US); R. Hollis WHITTINGTON, Portland, OR (US)

(73) Assignee: BIOTRONIK SE & CO. KG, Berlin (DE)

(21) Appl. No.: 19/131,079

(22) PCT Filed: Nov. 10, 2023
(86) PCT No.: PCT/EP2023/081377

§ 371 Date: May 19, 2025

Related U.S. Application Data
(60) Provisional application No. 63/429,628, filed on Dec. 2, 2022.

(30) Foreign Application Priority Data
Jan. 17, 2023 (EP) 23151916.6

(51) Int. Cl. A61B 5/364; A61B 5/00

(52) U.S. Cl. CPC A61B 5/364; A61B 5/686; A61B 5/7264; A61B 5/7282

(57) ABSTRACT

An implantable medical device for sensing electrocardiogram signals includes an arrangement of electrode poles configured to sense electrocardiogram signals and a processing module for processing electrocardiogram signals obtained by the arrangement of electrode poles. The processing module is configured to identify, based on said electrocardiogram signals obtained by the arrangement of electrode poles, premature ventricular contraction events over a prolonged period of time and to compute a burden measure indicative of the occurrence of premature ventricular contraction events in at least a portion of said prolonged period of time.

### CROSS-REFERENCE TO RELATED APPLICATIONS

This application is the United States National Phase under 35 U.S.C. § 371 of PCT International Patent Application No. PCT/EP2023/081377, filed on Nov. 10, 2023, which claims the benefit of European Patent Application No. 23151916.6, filed on Jan. 17, 2023, and U.S. Provisional Ser. No. 63/429,628, filed on Dec. 2, 2022, the disclosures of which are hereby incorporated by reference herein in their entireties.

### TECHNICAL FIELD

The instant invention generally relates an implantable medical device for sensing electrocardiogram signals and to a method for operating such an implantable medical device.

### BACKGROUND

An implantable medical device of the type concerned herein comprises an arrangement of electrode poles configured to sense electrocardiogram signals and a processing module configured for processing electrocardiogram signals obtained by the arrangement of electrode poles.

An implantable medical device of this kind may for example be a pacemaker, an implantable cardioverter defibrillator, a sensor device such as a bio-sensor, or a monitoring device. The implantable medical device herein is configured to sense electrocardiogram signals.

For example, the implantable medical device may be a monitoring device which is configured to record electrocardiogram signals and to communicate recorded electrocardiogram signals or information derived from recorded electrocardiogram signals to an external device in the context of a home monitoring system.

An implantable medical device as for example described in EP 3 278 836 B1 may for example comprise a housing and an arrangement of electrode poles arranged on the housing. The electrode poles herein are arranged on the housing of the implantable medical device such that the electrode poles are aligned along a longitudinal axis along which the implantable medical device extends. The electrode poles may for example be formed by housing segments which are made from an electrically conductive material such as a metal material and are exposed to the outside such that they may be brought into electrical contact with surrounding tissue in order to establish an electrical coupling to the tissue in an implanted state of the implantable medical device.

An implantable medical device as e.g. used in a home monitoring system shall allow for a reliable monitoring of a physiological state of a patient. In particular, using the implantable medical device it shall be possible to reliably detect an abnormal cardiac state based on recorded electrocardiogram signals. If an abnormality is detected in electrocardiogram signals, the implantable medical device shall be enabled to communicate with for example an external device of a home monitoring system, e.g. in order to trigger a message to a service center to alert medical personnel of a potential need for attention.

An abnormal cardiac state may in particular relate to the occurrence of premature ventricular contractions. Premature ventricular contraction events (in short PVCs; also referred to as premature ventricular complexes, premature ventricular beats, premature ventricular depolarizations, or ventricular extrasystoles) are generally triggered from the ventricular myocardium and can be associated with structural heart disease and with many forms of cardiac disease, independent of severity. Premature ventricular contractions may be a precursor of cardiac pathology, but are also known to accompany extra-cardiac pathologies such as uncontrolled hypertension, thyroid dysfunction, pulmonary disease and sleep apneas. In the absence of a diagnosed underlying cardiovascular pathology, premature ventricular contractions are termed idiopathic premature ventricular contractions. Tracking the incidence of premature ventricular contractions and long-term monitoring of a daily premature ventricular contraction burden may represent helpful diagnostic tools for physicians.

In the article by Marcus, Gregory M. entitled “Evaluation and management of premature ventricular complexes.” Circulation 141.17 (2020): 1404-1418 it is described how a premature ventricular contraction burden of a patient may be computed based on the identification of premature ventricular contraction events.

The present disclosure is directed toward overcoming one or more of the above-mentioned problems, though not necessarily limited to embodiments that do.

### SUMMARY

It is an object to provide an implantable medical device and a method for operating such an implantable medical device which in a reliable manner allow for a monitoring of a patient's cardiac condition over a prolonged period of time.

In one aspect, an implantable medical device for sensing electrocardiogram signals comprises an arrangement of electrode poles configured to sense electrocardiogram signals and a processing module for processing electrocardiogram signals obtained by the arrangement of electrode poles. The processing module is configured to identify, based on said electrocardiogram signals obtained by the arrangement of electrode poles, premature ventricular contraction events over a prolonged period of time and to compute a burden measure indicative of the occurrence of premature ventricular contraction events in at least a portion of said prolonged period of time.

Premature ventricular contraction properties can be highly variable, depending on the origin location and timing of the ventricular depolarization, but generally share two characteristics. A first characteristic is abnormal timing: Premature ventricular contractions occur prematurely, faster than the predominant sinus rhythm, and are typically followed by a compensatory pause due to retrograde block in the AV node preventing conduction of the next P-wave. A second characteristic relates to ECG morphology: The shape of a waveform relating to a premature ventricular contraction event is dependent on the origin location of the premature ventricular contraction, but the morphology is generally abnormal, and can be recognized when compared to a normally conducted beat. The goal of the current disclosure is to create a mechanism within an implantable medical device to discriminate premature ventricular contractions from normal beats, and to provide physicians with a measure of a premature ventricular contraction burden for diagnostic purposes.

In the instant solution, the implantable medical device comprises a processing module which is configured to process sensed electrocardiogram signals in order to identify premature ventricular contraction events within the electrocardiogram signals and to compute, based on the identified premature ventricular contraction events, a burden measure indicative of the occurrence of premature ventricular contraction events in at least a portion of a prolonged period of time. For example, over the lifetime of the implantable medical device, which may range from a multiplicity of months to a multiplicity of years and during which the implantable medical device rests in an implanted state within the patient, the implantable medical device senses electrocardiogram signals and, based on the sensed electrocardiogram signals, identifies premature ventricular contraction events in the electrocardiogram signals. From the identified premature ventricular contraction events, a burden measure is determined, which indicates e.g. a frequency of occurrence of premature ventricular contraction events for a patient and which may be put in relation to other measures, such as a given time period or the like.

The implantable medical device itself hence is configured to identify premature contraction events and to process premature ventricular contraction events to derive a burden measure indicating a load on the patient carrying the implantable medical device by the occurrence of premature ventricular contractions. The implantable medical device may communicate information relating to premature ventricular contraction events or the computed burden measure to an external device configured to operate outside of the patient, for example within the context of a home monitoring system, such that information relating to a premature ventricular contraction burden may be output and may be brought to the attention of a physician.

In one embodiment, the processing module is configured to continuously identify premature ventricular contraction events in a series of successive heartbeats over the prolonged period of time. For example, the implantable medical device continuously, during the prolonged period of time, corresponding for example to the lifetime of the implantable medical device, senses ventricular contraction events, processes such ventricular contraction events and classifies ventricular contraction events, according to predefined characteristics and conditions, as premature ventricular contraction events or not. From the continuous monitoring of ventricular contraction events and from the identification of premature ventricular contraction events based on the monitoring, a burden measure of the patient may be recorded, indicating for example the number, percentage or frequency of premature ventricular contractions per hour, per day or for another given period of time, and may be output for example by communicating the burden measure to an external device such that a continuous record of a burden measure over time is obtained.

In one embodiment, the processing module is configured to compute, as the burden measure, at least one of a value indicative of a number of premature ventricular contraction events in a given time period, a value indicative of the percentage of premature ventricular contraction events in a total number of heartbeats, a value indicative of the maximum number of premature ventricular contraction events in a first time period within a given, second time period, or a value indicative of a number of polymorphic beats in a given time period.

The burden measure may in particular be computed as the number of premature ventricular contractions in a given time period, e.g. per 24 hours or per hour. For example, based on this, a burden measure may be computed to correspond to an average number of premature ventricular contractions per day over the last week or last month or in another e.g. user-configurable time period.

Alternatively or in addition, the burden measure may be computed to correspond to the number of premature ventricular contractions as a percentage value of total beats, e.g. during different periods of arrhythmia or for different types of arrhythmia.

Yet alternatively or in addition, the burden measure may be computed to correspond to the maximum number of premature ventricular contractions in a given time period, e.g. the maximum number of premature ventricular contractions in a 1-hour time period within one day.

Yet alternatively or in addition, the burden measure may be computed to correspond to the number of polymorphic beats, i.e. premature ventricular contractions with varying morphology, or the premature ventricular contraction burden of beats with varying morphology.

In one embodiment, the processing module is configured to compute the burden measure in relation to at least one further measure. Hence, the burden measure is put in relation to another quantity, such that the occurrence of premature ventricular contractions is put into context with other information available to the implantable medical device.

For example, the at least one further measure may include the time of day, a respiratory measure, an activity measure, an environmental measure, and/or a pharmacological measure.

For example, in one embodiment, the burden measure may be computed and recorded based on environmental or pharmacological changes. For example, if it is detected by the implantable medical device or if the implantable medical device is informed by another implanted or external device that a patient undergoes a certain environmental or pharmacologic condition, this may be put into context and stored together with the recorded burden measure. For example, periods of increased stress or increased alcohol use or caffeine use or periods of warm weather or cold weather or specific pharmacotherapy changes may be put into context and stored together with the recorded burden measure.

In another example, the implantable medical device may detect a period of increased activity of the patient, for example based on a sensor output of a motion sensor included in the implantable medical device or in another device carried by the patient and being in communication connection with the implantable medical device. The implantable medical device may then be configured to record the burden measure during periods of activity and during periods after activity as compared with a baseline burden measure not related to activity, for example during periods of sleep.

In yet another example, a respiratory information may be recorded by the implantable medical device, the respiratory information relating to a respiratory state of the patient. In this way, the recorded burden measure may be put into relation to and stored together with respiratory information, for example during overnight periods with normal respiration vs. periods with sleep disordered breathing.

In yet another example, the burden measure may be recorded by time of day to determine circadian variations and correlations with lifestyle factors such as smoking, drinking alcohol, waking, and other factors. The recording of the burden measure by time of day may also include time-of-day long-term trends over multiple days, months or years. For instance, this allows for a tracking of the burden measure during certain time periods over a prolonged period of time, e.g. between 8:00 AM and 9:00 AM over a six month timeframe.

In one embodiment, the implantable medical device is configured to communicate information relating to the burden measure to an external device. The implantable medical device may for example periodically or in an event-driven manner report information about the burden measure to the external device, for example within the context of a home monitoring system.

For example, if it is found that the recorded burden measure crosses a certain threshold, for example 1000 premature ventricular contractions in a 24 hour time period, this may suggest an increased risk of sudden cardiac death. If it is found that the computed burden measure crosses a predefined threshold, this may trigger an alert message by the implantable medical device to the external device, such that a physician may be notified of a potentially hazardous cardiac state of the patient.

In another example, a higher value for the burden measure, for example a value larger than e.g. 0.1% premature ventricular contractions per 24 hr, in a patient with heart failure (HF) may be associated with a worsening HF and increased risk of cardiac events, and may trigger a warning message communicated by the implantable medical device to an external device in order to trigger a physician to more closely monitor a cardiac condition of the patient or change therapy of the patient.

In one embodiment, the processing module is configured to identify a ventricular contraction event based on said electrocardiogram signals, compute at least one discrimination metric value for said ventricular contraction event, compare said at least one discrimination metric value to at least one of a first reference value computed based on a first number of prior ventricular contraction events and a second reference value computed based on a second number of subsequent ventricular contraction events, and classify said ventricular contraction event as a premature ventricular contraction event based on said comparison.

This makes use of the fact that for a premature ventricular contraction event it can be assumed that a waveform relating to the premature ventricular contraction event comprises an abnormal shape, in comparison to other, regular ventricular contraction events. Hence, within the implantable medical device, a premature ventricular contraction event shall be identified by assessing the morphology of a waveform relating to a particular ventricular contraction event. If it is found that abnormalities exist in a particular ventricular contraction event indicating a premature ventricular contraction event, the particular ventricular contraction event in question shall be classified as a premature ventricular contraction event.

In particular, the processing module is configured to identify a ventricular contraction event based on sensed electrocardiogram signals and to compute one or multiple discrimination metric values relating to the identified ventricular contraction event. The one or the multiple discrimination metric values are compared to one or multiple first reference values computed based on a first number of prior ventricular contraction events and/or to one or multiple second reference values computed based on a second number of subsequent ventricular contraction events. Based on the comparison, the ventricular contraction event is classified as a premature ventricular contraction event (or not).

Hence, one or multiple discrimination metric values are computed. The discrimination metric values relate to the morphology of a waveform of the ventricular contraction event which is currently assessed. The one or the multiple discrimination metric values are compared to reference values, and based on the comparison it is identified whether the discrimination metric values indicate an abnormal waveform possibly indicative of a premature ventricular contraction event (or not).

Herein, the one or the multiple discrimination metric values are compared to one or multiple reference values relating to prior ventricular contraction events, which generally can be assumed to be regular ventricular contraction events of regular heartbeats, and/or to one or multiple reference values relating to subsequent ventricular contraction events, which also generally can be assumed to relate to regular heartbeats. The reference values hence are determined based on prior ventricular contraction events and/or subsequent ventricular contraction events.

Each reference value should indicate a value for a discrimination metric value in question which is indicative of a normal state and hence a normal ventricular contraction event of a regular heartbeat. If, by the comparison of the discrimination metric value computed for the instant ventricular contraction event, it is found that the discrimination metric value differs from the reference value e.g. by more than a certain margin, it is identified that an abnormal waveform exists exhibiting an abnormal morphology, such that the ventricular contraction event may be classified as a premature ventricular contraction event.

The first number of prior ventricular contraction events, based on which the first reference value is determined, may for example lie in a range between 2 to 50, for example 3 to 20, for example 6 prior ventricular contraction events. The prior ventricular contraction events may be successive ventricular contraction events immediately prior to the instantly assessed ventricular contraction event, or may be non-successive.

The second number of subsequent ventricular contraction events, based on which the second reference value is determined, may for example lie in a range between 2 to 50, for example 3 to 20, for example 6 subsequent ventricular contraction events. The subsequent ventricular contraction events may be successive ventricular contraction events immediately subsequent to the instantly assessed ventricular contraction event, or may be non-successive.

The first number may be equal to the second number or may differ from the second number.

The classification of a premature ventricular contraction event generally takes place, in particular if a second reference value relating to subsequent ventricular contraction events is taken into account, with a time delay to cover a time period within which the subsequent ventricular contraction events are recorded.

In one embodiment, the processing module is configured to compute the at least one discrimination metric value, with relation to a signal portion relating to the ventricular contraction event, based on at least one of the following: a maximum positive amplitude, a maximum negative amplitude, a maximum rectified amplitude, a peak-to-peak amplitude, a maximum first derivative value, a maximum second derivative value, an area value under a curve of said signal portion until a first zero-crossing, an area value under a curve of said signal portion between a first zero-crossing and a second zero crossing, an area value under a curve of said signal portion between a second zero-crossing and a third zero crossing, a time duration value until a first zero-crossing, a time duration value between a first zero-crossing and a second zero crossing, a time duration value between a second zero-crossing and a third zero crossing and/or a time duration value between an upward crossing and a downward crossing of a ventricular detection threshold.

One or multiple quantities may be computed, accordingly, as one or multiple discrimination metric values. The discrimination metric value may for example be computed according to the maximum positive amplitude, the maximum negative amplitude, a maximum rectified amplitude, or a peak-to-peak amplitude of a waveform relating to a currently assessed ventricular contraction event. Alternatively or in addition, a discrimination metric value may be computed according to a maximum value of a first derivative or a second derivative of the waveform relating to the currently assessed ventricular contraction event. Alternatively or in addition, the discrimination metric value may be computed according to an area value indicative of an area under a curve relating to the currently assessed ventricular contraction event, wherein the area value may relate to the area until the first zero-crossing, to an area between the first zero-crossing and a second zero-crossing, or to an area between the second zero-crossing and a third zero-crossing. Alternatively or in addition, the discrimination metric value may be computed to be indicative of a time duration, for example between the initial detection of the ventricular contraction event and a first zero-crossing, between the first zero-crossing and a second zero-crossing, or between the second zero-crossing and a third zero-crossing, or between an upward crossing of a ventricular detection threshold, based on which the ventricular detection event is initially identified, and a downward crossing of the ventricular detection threshold.

Generally, one or multiple discrimination metric values may be computed, wherein any combination of discrimination metric values may be used to identify a premature ventricular contraction event. Each discrimination metric value herein is compared to at least one of an associated first reference value and an associated second reference value, the particular reference value being indicative of a normal value for the particular discrimination metric value in question.

Each reference value may be computed by applying a statistical analysis of prior ventricular contraction events and/or subsequent ventricular contraction events. In particular, the first reference value may be computed based on a first statistical measure relating to the first number of prior ventricular contraction events. In turn, the second reference value may be computed based on a second statistical measure relating to the second number of subsequent ventricular contraction events. The first reference value hence is determined by statistical analysis of prior ventricular contraction events. In contrast, the second reference value is determined by statistical analysis of subsequent ventricular contraction events.

The particular reference value in particular may be computed according to any standard statistical quantity obtained by statistical analysis. For example, the first reference value and/or the second reference value may be computed according to a mean value, a standard deviation value, a coefficient of variation, a Shannon entropy value, an exponential moving average value e.g. according to a function with varying beats/varying weights associated with the surrounding beats, a median value, a percentile value, e.g. a 5% to 95% percentile value, a skew value, a kurtosis value, and/or a root mean square value e.g. of successive differences. If for example the discrimination metric value in question is computed according to the maximum amplitude of the waveform relating to the current ventricular contraction event, the reference value is determined by e.g. an averaging or by another statistical measure of the maximum amplitude values of prior ventricular contraction events and/or subsequent ventricular contraction events. If the discrimination metric value in question is an area value relating to the waveform of the current ventricular contraction event or a time duration value, the reference value is determined by a statistical measure relating to the associated area value or time duration value of prior ventricular contraction events and/or subsequent ventricular contraction events.

In one embodiment, the processing module is configured to classify the ventricular contraction event which is currently assessed as a premature ventricular contraction event if the at least one discrimination metric value deviates from the first reference value by more than a first margin and/or deviates from the second reference value by more than a second margin. For classifying a currently assessed ventricular contraction event as a premature ventricular contraction event, the processing module compares one or multiple computed discrimination metric values to one or multiple reference values relating to prior ventricular contraction events and/or subsequent ventricular contraction events. If it is found that the one or the multiple discrimination metric values differ from associated reference values, this is interpreted to indicate that the waveform of the currently assessed ventricular contraction event is abnormal in that it differs from a waveform of a regular ventricular contraction event, and accordingly the ventricular contraction event may be classified as a premature ventricular contraction event (wherein potentially further conditions may be taken into account).

For example, the processing module is configured to determine the first margin based on a percentage value of the first reference value and/or to determine the second margin based on a percentage value of the second reference value. The first reference value and the second reference value are dynamically determined based on a number of prior ventricular contraction events and/or based on a number of subsequent ventricular contraction events. Based on the current value for the first reference value and/or the second reference value the first margin and/or the second margin are set. The percentage value herein may be fixed, e.g. in a range between 1% to 50% or the like. In another embodiment the percentage value may be dynamically adapted, for example based on the current value of the first reference value and/or the second reference value.

In one embodiment, the processing module is configured to compute multiple discrimination metric values for the ventricular contraction event and to compare the multiple discrimination metric values to multiple first reference values computed based on the first number of prior ventricular contraction events and/or multiple second reference values computed based on the second number of subsequent ventricular contraction events. Hence, multiple discrimination metric values are determined for the currently assessed ventricular contraction event and are compared to associated first and/or the second reference values. Based on the comparison of the multiple discrimination metric values to the associated reference values, the currently assessed ventricular contraction event is classified as a premature ventricular contraction event (or not).

In one embodiment, the processing module is configured to classify the ventricular contraction event as a premature ventricular contraction event if, based on the comparison, at least for a subset of the discrimination metric values a set of predefined conditions is fulfilled. Within the comparison, the computed discrimination metric values are compared to the associated reference values. If at least for some discrimination metric values the comparison yields that the discrimination metric values for example differ from the associated reference values in each case by more than a certain margin, the currently assessed ventricular contraction event may be classified as a premature ventricular contraction event. For example, it may be generally required that for two out of three discrimination metric values the comparison yields that the discrimination metric values differ from the associated reference values by more than a certain margin, such that for two out of three discrimination metric values an associated condition is fulfilled.

In one embodiment, the processing module is configured to classify the ventricular contraction event as a premature ventricular contraction event based on the comparison and in addition based on a first timing distance between the ventricular contraction event and an immediately prior ventricular contraction event and/or a second timing distance between the ventricular contraction event and an immediately subsequent ventricular contraction event. Generally, for a premature ventricular contraction event, the morphology of a waveform relating to the premature ventricular contraction event differs from the morphology of a regular ventricular contraction event. This is assessed by means of comparing discrimination metric values to associated reference values. In addition, it is characteristic for a premature ventricular contraction event that the contraction event comes at a relatively short timing distance after a prior ventricular contraction event and is followed by a lengthy pause before another, regular ventricular contraction event occurs. Hence, the timing of the currently assessed ventricular contraction event may be evaluated in order to define an additional, necessary condition which must be fulfilled in order to classify the currently assessed ventricular contraction event as a premature ventricular contraction event. If for example the first timing distance between the ventricular contraction event and the immediately prior ventricular contraction event is smaller than a first timing threshold and/or the second timing distance between the ventricular contraction event and the immediately subsequent ventricular contraction event is larger than a second timing threshold, and if in addition the assessment of at least one discrimination metric value computed based on the currently assessed ventricular contraction event yields an abnormality, the currently assessed ventricular contraction event may be classified as a premature ventricular contraction event. Hence, not only the morphology is evaluated, but in addition also a timing of the ventricular contraction event is taken into account for classifying the ventricular contraction event as a premature ventricular contraction event.

The implantable medical device may, in one embodiment, comprise multiple electrode poles, which for example are aligned along a longitudinal axis and hence are arranged at different axial positions on the implantable medical device. By means of the different electrode poles an electrical coupling to surrounding tissue is established when the implantable medical device is implanted in a patient, such that electrocardiogram signals may be sensed using the different electrode poles.

In another aspect, a method for operating an implantable medical device for sensing electrocardiogram signals comprises: sensing electrocardiogram signals using an arrangement of electrode poles of the implantable medical device; and processing electrocardiogram signals obtained by the arrangement of electrode poles using a processing module of the implantable medical device. The processing module identifies, based on said electrocardiogram signals obtained by the arrangement of electrode poles, premature ventricular contraction events over a prolonged period of time and computes a burden measure indicative of the occurrence of premature ventricular contraction events in at least a portion of said prolonged period of time.

The advantages and advantageous embodiments described above for the system equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily under-stood with reference to the following detailed description and the embodiments shown in the drawings. Herein,

FIG. 1 shows a schematic drawing of an implantable medical device implanted in a patient;

FIG. 2 shows a schematic drawing of an embodiment of an implantable medical device comprising an arrangement of electrode poles;

FIG. 3 shows a schematic drawing of another embodiment of an implantable medical device;

FIG. 4 shows a waveform relating to a premature ventricular contraction event;

FIG. 5 shows a series of ventricular contraction events;

FIG. 6 shows a premature ventricular contraction event within a series of ventricular contraction events;

FIG. 7 shows a graph of a burden measure as recorded over a prolonged period of time; and

FIG. 8 shows graphs of a burden measure for different patients prior to, during and after a period of exercise.

### DETAILED DESCRIPTION

Subsequently, embodiments of the invention shall be described in detail with reference to the drawings. In the drawings, like reference numerals designate like structural elements.

It is to be noted that the embodiments are not limiting for the invention, but merely represent illustrative examples.

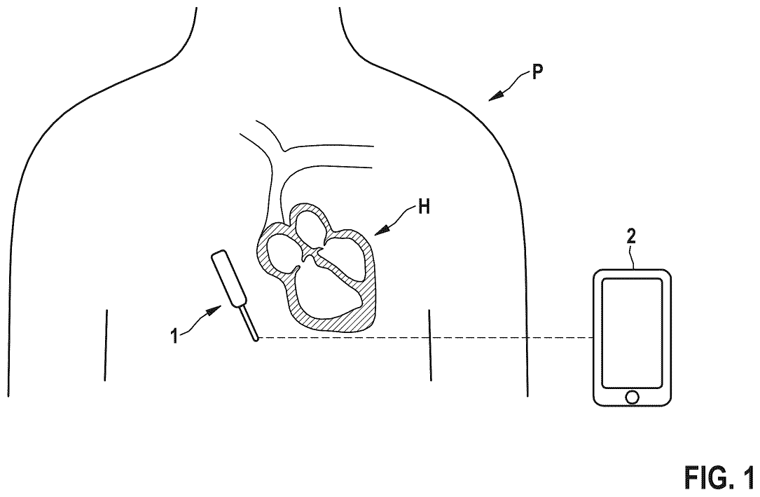

Referring to FIG. 1, in one embodiment a system comprises an implantable medical device 1 implanted (for example subcutaneously) into a patient for serving a therapeutic and/or diagnostic function. The implantable medical device 1 may for example be implanted subcutaneously into a patient P for monitoring cardiac activity of the patient's heart H. The implantable medical device 1, for this, comprises an arrangement of electrode poles which are used to couple to surrounding tissue and to sense electrocardiogram signals originating from the heart H.

The system furthermore comprises an external device 2 external to the patient P and being in communication connection with the implantable medical device 1.

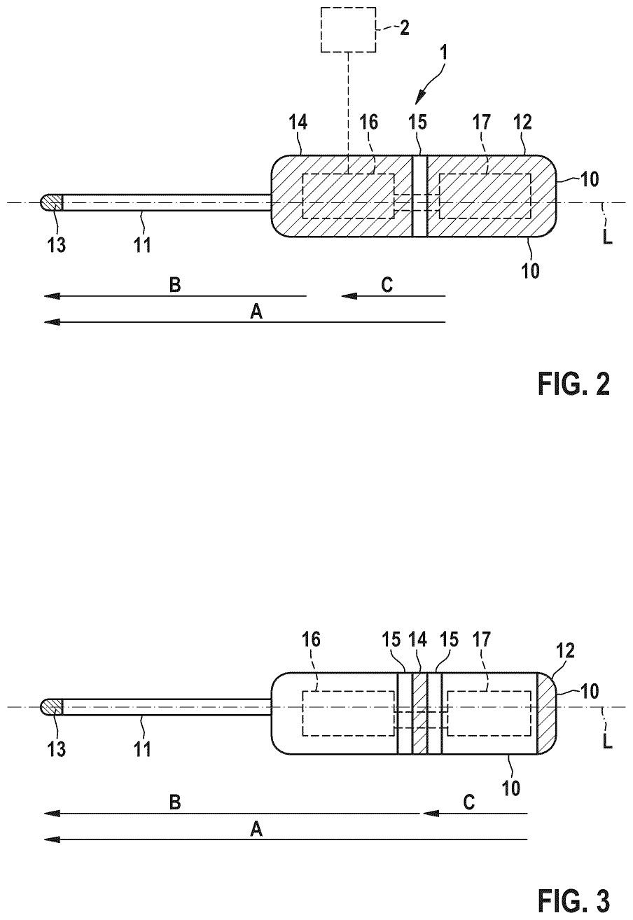

Referring now to FIG. 2, in one embodiment the implantable medical device 1 comprises a housing 10 formed e.g. by different housing segments, the housing 10 enclosing and encapsulating a processing module 16 formed by electronic circuitry and a battery module 17. In particular, a first housing segment may receive and enclose the processing module 16, whereas a second housing segment receives and encloses the battery module 17. Another housing segment 11 longitudinally extends from the first and second housing segments and forms a header portion having reduced cross-sectional dimensions with respect to the other housing segments.

In the embodiment of FIG. 2, a first electrode pole 12 is formed by the housing segment enclosing the battery module 17, a second electrode pole 13 is arranged at a far end of the housing segment 11 forming the header portion, and a third electrode pole 14 is formed by the housing segment enclosing the processing module 16. The implantable medical device 1 with its housing 10 generally extends along a longitudinal axis L, the electrode poles 12, 13, 14 being aligned along the longitudinal axis L and being axially displaced with respect to one another along the longitudinal axis L. The electrode poles 12, 13, 14 herein are electrically separated from one another, an electrically insulating segment 15 being arranged in between the electrode poles 12, 14 formed on the main housing portion and the header portion formed by the housing segment 11 separating the electrode pole 13 from the other two electrode poles 12, 14.

In the embodiment of FIG. 2, the electrode poles 12, 13, 14 may be formed by portions of the housing 10 itself, the housing 10 being made for example from an electrically conductive material, in particular a metal material. By exposing portions of the housing 10 towards the outside, the electrode poles 12, 13, 14 are formed and may electrically contact with surrounding tissue in order to establish a coupling between the electrode poles 12, 13, 14 to the surrounding tissue.

Referring now to FIG. 3, in another embodiment the first electrode pole 12 is formed at an end of a housing segment of the housing 10 encapsulating the battery module 17, whereas the electrode pole 14 is formed by an electrode element which is electrically insulated from other portions of the housing 10 by insulating segments 15. For example, a multilayered pole element may be employed for forming the electrode pole 14, as it is described for example in EP 3 278 836 B1. The electrode pole 13 again is formed at a far end of the housing segment 11 forming the header portion.

In any of the embodiments of FIGS. 2 and 3, using the arrangement of electrode poles 12, 13, 14, electrocardiogram signals may be received and processed by the processing module 16. Based on the processing, a communication with an external device 2 may be established, for example to transmit alert messages to the external device 2 for example within the context of a home monitoring system for monitoring a physiological state of the patient P.

The different electrode poles 12, 13, 14 herein define signal reception vectors A, B, C by means of which electrocardiogram signals may be received using pairs of associated electrode poles 12, 13, 14. In particular, a first signal reception vector A is formed between the first electrode pole 12 and the second electrode pole 13, a second signal reception vector B is formed between the third electrode pole 14 and the first second electrode pole 13, and a third signal reception vector C is formed between the first electrode pole 12 and the third electrode pole 14. As the first electrode pole 12 and the second electrode pole 13 are arranged at opposite ends of the housing 10, the associated signal reception vector A is longer than the other two signal reception vectors B, C.

The different electrode poles 12, 13, 14 form different pairs of electrode poles 12, 13, 14 spanning different signal reception vectors A, B, C. By means of the different signal reception vectors A, B, C different electrocardiogram signals may be received and may be processed in a multi-channel processing.

It shall be noted herein that the implantable medical device 1 may be a monitoring device (as schematically shown in FIGS. 2 and 3), a pacemaker device, a defibrillator device or any other implantable medical device configured for implantation into a patient P. The instant text in particular is not limited to a monitoring device configured for implantation outside of a patient's heart H.

The implantable medical device 1 as described herein shall generally be configured to classify premature ventricular contraction events such that for example a premature ventricular contraction burden of a patient P may be computed, for example indicating the number of premature ventricular contraction events per day.

Generally, it is characteristic for a premature ventricular contraction event that the premature ventricular contraction event comes at a premature, short timing distance after a prior ventricular contraction event and is followed by a comparatively lengthy pause before another, subsequent ventricular contraction event occurs. In addition, a premature ventricular contraction event generally exhibits a waveform which in its morphology substantially differs from the waveform of a regular ventricular contraction event.

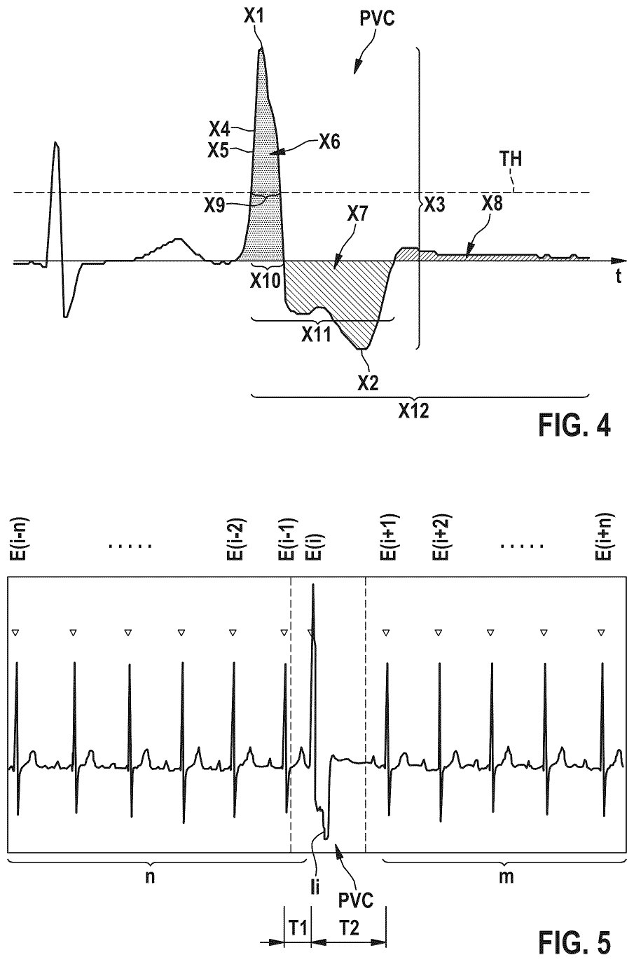

Referring now to FIG. 4, a premature ventricular contraction event PVC comprises a morphology different than a regular ventricular contraction event of a regular sinus rhythm of the patient's heart H. The morphology herein may be characterized by certain discrimination metrics, such as a maximum positive amplitude X1, a maximum negative amplitude X2, a maximum peak-to-peak amplitude X3, a maximum slope value X4, an maximum value of the second derivative X5, an area X6 under a positive R peak prior to a first zero-crossing X6, an area X7 under the curve between a first zero-crossing and a second zero-crossing, an area X8 under the curve between the second zero-crossing and a third zero-crossing, a time duration X9 between an upward crossing and a downward crossing of a ventricular detection threshold TH, a time duration X10 between the upward crossing of the ventricular detection threshold TH and the first zero-crossing, a time duration X11 between the upward crossing of the ventricular detection threshold TH and the second zero-crossing, and/or a time duration X12 between the upward crossing of the ventricular detection threshold TH and a third zero-crossing.

For a particular ventricular contraction waveform in question, values for all or some of the discrimination metrics may be computed and may be assessed in order to identify whether an abnormal morphology potentially indicative of a premature ventricular contraction event is present.

To classify a ventricular contraction event as a premature ventricular contraction event, in one embodiment at least one discrimination metric value X1 . . . X12 is computed by the processing module 16 of the implantable medical device 1. The at least one discrimination metric value is then compared to at least one reference value, and based on the comparison the ventricular contraction event in question is classified as a premature ventricular contraction waveform (or not).

Referring now to FIG. 5, reference values may in particular be determined according to a number n of prior ventricular contraction events E(i−n) . . . E(i−1) and/or a number m of subsequent ventricular contraction events E(i+1) . . . E(i+m).

In particular, for a specific discrimination metric value, such as the maximum positive amplitude X1 or the area X6 under the positive R peak prior to the first zero-crossing, an associated first reference value may be computed based on the associated discrimination metric for the number n of prior ventricular contraction events E(i−n) . . . E(i−1). Alternatively or in addition, a second reference value may be computed based on the associated discrimination metric for the number m of subsequent ventricular contraction events E(i+1) . . . E(i+m).

The particular reference value may in particular be computed according to a statistical measure by applying a statistical analysis. For example, the particular reference value may correspond to a mean value, a standard deviation value, a coefficient of variation, a Shannon entropy value, an exponential moving average value, a median value, a percentile value, a skew value, a kurtosis value, or a root mean square value relating to the particular discrimination metric X1 . . . X12.

For example, if the maximum positive amplitude X1 is assessed as the discrimination metric, the first reference value may be determined by averaging the maximum positive amplitude values of the n prior ventricular contraction events E(i−n) . . . E(i−1), and the second reference value may be determined by averaging the maximum positive amplitude values of the m subsequent ventricular contraction events E(i+1) . . . E(i+m).

Making use of the reference values, then, the particular ventricular contraction event in question may be classified as a premature ventricular contraction event if for example a particular discrimination metric value as computed for the ventricular contraction event differs by more than a certain margin from the respective reference value. For example, if both a first reference value relating to prior ventricular contraction events E(i−n) . . . E(i−1) and a second reference value relating to subsequent ventricular contraction events E(i+1) . . . E(i+m) is taken into account, a ventricular contraction event may be classified as a premature ventricular contraction event if the discrimination metric value differs by more than a first margin from the first reference value and by more than a second margin from the second reference value.

If multiple discrimination metric values relating to different discrimination metrics are computed, multiple different first reference values and/or or multiple different second reference values may be taken into account, the different reference values relating to the different discrimination metrics. Herein, the ventricular contraction event may be classified as a premature ventricular contraction event if at least for a subset of the discrimination metric values an associated set of conditions is fulfilled. For example, it may be found for a premature ventricular contraction event if for two out of three of the discrimination metric values it is found that the particular discrimination metric value differs from an associated reference value by more than a certain margin.

The margin, in each case, may be for example computed based on a percentage of the particular reference value, wherein the percentage may be fixed or may be dynamically adapted during operation of the system. In the example of FIG. 5, for the ventricular contraction event E(i) it for example is found that based on one or multiple discrimination metric values relating to one or multiple different discrimination metrics a substantial deviation of the morphology in comparison to prior ventricular events E(i−n) . . . E(i−1) and/or to subsequent ventricular events E(i+1) . . . E(i+m) is present, such that the ventricular contraction event E(i) is classified as a premature ventricular contraction event PVC.

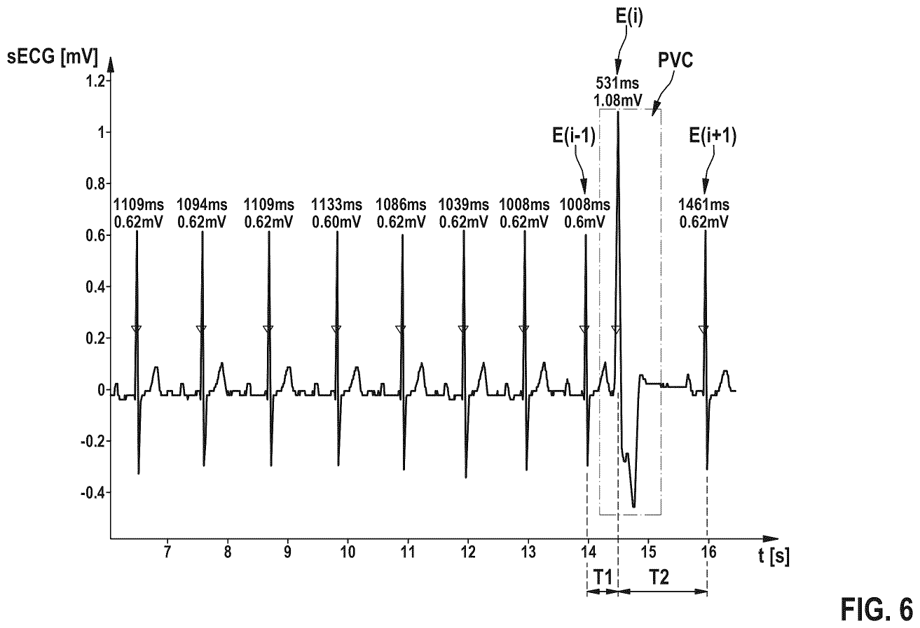

Generally, a premature ventricular contraction event PVC not only substantially differs in the morphology of the associated waveform, but also in the timing from a regular contraction event. Hence, in addition, it may be assessed whether a timing distance Tl of the ventricular contraction event E(i) is smaller than a first timing threshold, hence indicating that the ventricular contraction event E(i) occurs prematurely with respect to a prior ventricular contraction event E(i−1), as indicated in FIG. 6. In addition, it may be assessed whether a timing distance T2 of the ventricular contraction event E(i) is larger than a second timing threshold, hence indicating that after the ventricular contraction event E(i) a substantial pause occurs, longer than the length of a regular heartbeat, as visible in FIG. 6.

In order to provide for a reliable classification of premature ventricular contraction events PVC, hence, a timing and in addition a morphology may be assessed, wherein the combined assessment yields a classification of a current ventricular contraction event E(i) as a premature ventricular contraction event PVC if both a timing condition and a morphology condition is fulfilled.

For example, in one embodiment, an R-wave peak amplitude discrimination metric is used in combination with timing. If the peak amplitude of a given QRS complex is different from the mean of previous peak amplitudes by a given percentage and is also different from the mean of the following R-wave peak amplitudes by a second given percentage, and the interval preceding the given QRS complex is shorter than a given short interval threshold and the interval following the QRS complex is longer than a given long interval threshold, then the QRS complex is classified as a premature ventricular contraction event PVC. In this embodiment, the minimum change in R-wave amplitude may fall in the range of 2.5% to 50% of the preceding or following cycles'R-wave amplitude. The timing threshold for determining the short or premature interval may be determined in either a number of milliseconds or a percentage or the instantaneous or average cycle time. Furthermore, the threshold may be static as a programmable fixed number or could be a dynamic parameter that is adjusted based on the variation in the RR-intervals in the vicinity of the current QRS complex. The same principles may apply to the determination of the long interval threshold. The amplitude threshold may also be a static percentage (like 10%) or may be dynamically determined based on the amount of variation in previous or following signal amplitudes.

In another embodiment, similar principles are applied but a different discrimination metric is used. In this embodiment the critical quantity is the time-to-zero crossings. The individual parameters, or a combination of zero-crossing times X10, X11, X12 determined as the weighted sum of related time periods X10, X11, X12 are used as discrimination metrics in order to determine if the morphology and/or the total duration of the complex has changed. This final combination may be used alone or together with timing criteria as described in the first embodiment. Absolute thresholds or dynamic thresholds based on the properties of the signals from past cycles or next cycles may be used to determine if the instant ventricular contraction event is a premature ventricular contraction event PVC.

The described scheme allows discrimination of premature ventricular contraction events from normal beats and the recording of a burden measure, indicative of cardiac dysfunction, electrical abnormalities, and worsening heart failure in a patient. The solution may be achievable e.g. using existing functionality in an implantable medical device such as a monitoring device, namely the use of timing criteria and simple signal characteristics like R-wave amplitude, morphology, or duration. Due to the simplicity, on-board hardware generally does not need to be changed and does not consume significant power in addition to normal operation.

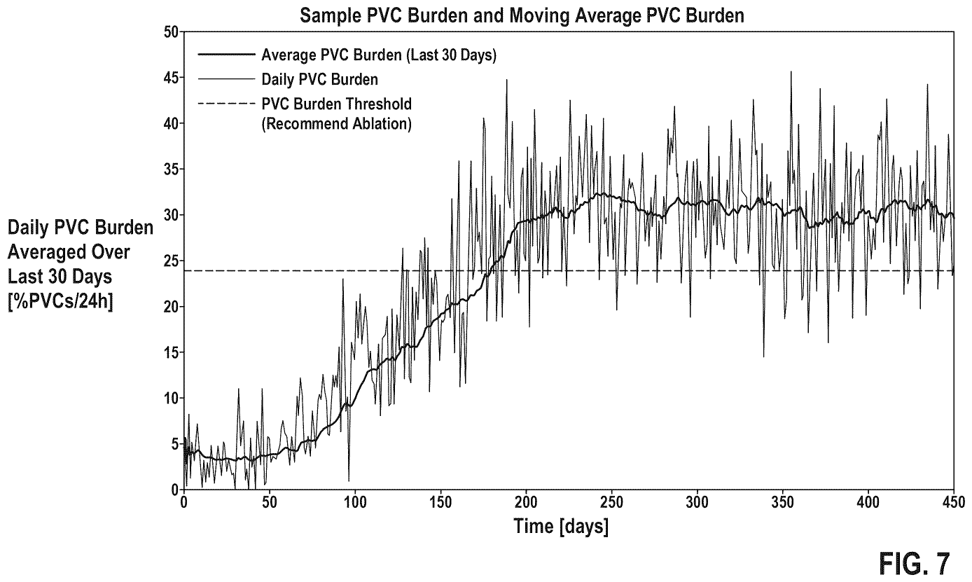

Furthermore, the classification criteria are explainable and consistent with clinical practice for identifying premature ventricular contractions. The premature ventricular contraction detection algorithm can generally be used to provide a premature ventricular contraction counter per day, or a premature ventricular contraction burden over the course of the day to stratify risk of morbidity and mortality in cardiac patients. Referring now to FIG. 7, the implantable medical device 1 is configured to identify premature ventricular contraction events and, based on the identification of premature contraction events, to compute a burden measure at least in a portion of a prolonged period of time, for example corresponding to the lifetime of the implantable medical device 1. In the graph of FIG. 7, a burden measure, such as the percentage of premature ventricular contractions in relation to the total number of heartbeats within a time period of 24 hours, is shown as continuously recorded over a time period of (about) 450 days. FIG. 7 herein shows the actual values of the burden measure and an average burden computed by a moving averaging using a moving time window of 30 days.

For recording the burden measure, the processing module 16 of the implantable medical device 1 is configured to identify premature ventricular contraction events based on sensed electrocardiogram signals and to compute, from the identified premature ventricular contraction events, the burden measure. For identifying the premature ventricular contraction events a scheme as outlined above may be used. It shall be noted, however, that any other algorithm, for example based on monitoring only a timing of ventricular contraction events, may be employed for identifying premature ventricular contraction events.

In general, a number of premature ventricular contractions in a given time period, a number of premature ventricular contractions as a percentage value of total beats, a maximum number of premature ventricular contractions in a first time period within a longer, second time period, or a number of polymorphic beats in a given timeframe may be determined as the burden measure.

In addition, the implantable medical device 1 may be configured to put the computed burden measure in relation to a further measure, such as the time of day, an environmental or pharmacological change, an activity measure, or a respiratory measure.

For example, making use of information for example provided by a motion sensor of the implantable medical device 1 or by another device carried by the patient, an activity measure may be recorded together with the burden measure such that the recorded burden measure is put in relation with and stored together with a state of activity of the patient.

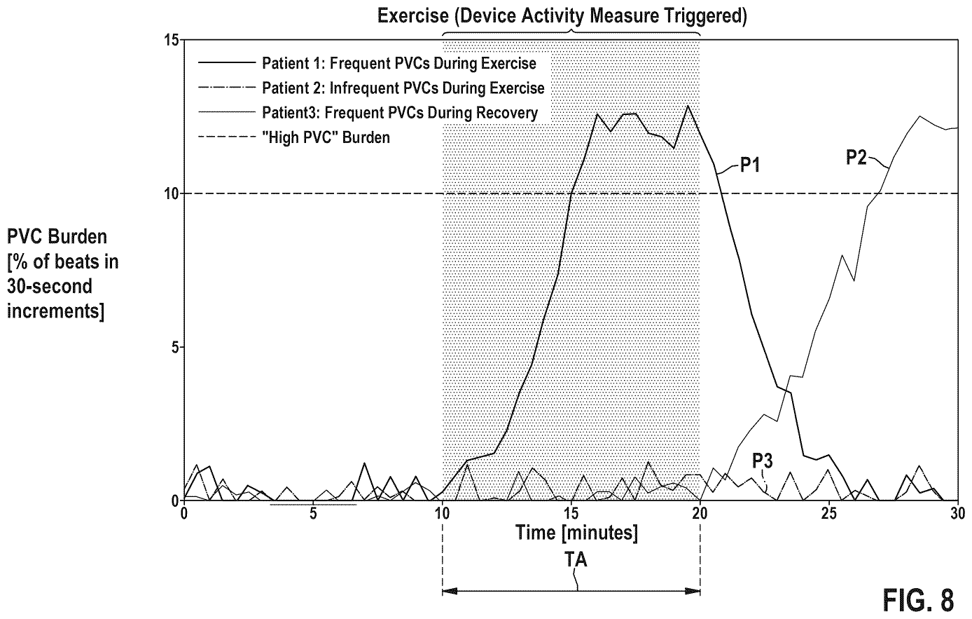

This is illustrated in FIG. 8, showing three graphs for three different patients P1, P2, P3 prior to a period of activity TA, during a period of activity TA, and after a period of activity TA. As is visible from FIG. 8, for different patients P1, P2, P3 a burden measure may behave differently in relation to a patient activity, one patient Pl for example exhibiting an increased burden measure during the period of activity TA, another patient P2 exhibiting an increased burden measure not during, but after a period of activity TA, and yet another patient P3 exhibiting no change in the burden measure at all.

The burden measure is continuously computed and recorded by the implantable medical device 1 and is for example communicated to an external device 2, which may further process information relating to the burden measure. A report of the burden measure may be periodically triggered by the implantable medical device 1, or may be triggered in an event-driven manner.

For example, the implantable medical device 1 may monitor whether the burden measure exceeds a certain threshold, for example 1000 premature ventricular contractions per day or 0.1% of premature ventricular contractions in the total number of beats in a 24-hour time period. Based on the monitoring, alert messages may be triggered and sent to the external device 2 in order to potentially trigger an alarm.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device
10 Housing
11 Housing segment (header portion)
12 Electrode pole
13 Electrode pole
14 Electrode pole
15 Electrically insulating segment
16 Processing module
17 Battery module
2 External device
A, B, C Signal reception vector
E(i−n) . . . E(i+m) Ventricular contraction event
H Heart
i Current beat
L Longitudinal axis
n Prior beats
m Subsequent beats
P Patient
PVC Premature ventricular contraction event
T1 Timing distance
T2 Timing distance
TA Period of activity
TH Ventricular detection threshold
X1 . . . X12 Discrimination metric value

1. An implantable medical device for sensing electrocardiogram signals, comprising:
an arrangement of electrode poles configured to sense electrocardiogram signals; and
a processing module for processing electrocardiogram signals obtained by the arrangement of electrode poles;
wherein the processing module is configured to
identify, based on said electrocardiogram signals obtained by the arrangement of electrode poles, premature ventricular contraction events over a prolonged period of time and
compute a burden measure indicative of the occurrence of premature ventricular contraction events in at least a portion of said prolonged period of time.

2. The implantable medical device according to claim 1, wherein the processing module is configured to continuously identify premature ventricular contraction events in a series of successive heart beats over said prolonged period of time.

3. The implantable medical device according to claim 1, wherein the processing module is configured to compute, as the burden measure, at least one of a value indicative of the number of premature ventricular contraction events in a given time period, a value indicative of the percentage of premature ventricular contraction events in a total number of heart beats, a value indicative of the maximum number of premature ventricular contraction events in a first time period within a given, second time period, or a value indicative of a number of polymorphic beats.

4. The implantable medical device according to claim 1, wherein the processing module is configured to compute said burden measure in relation to at least one further measure.

5. The implantable medical device according to claim 4, wherein said at least one further measure includes the time of day, a respiratory measure, an activity measure, an environmental measure, and/or a pharmacological measure.

6. The implantable medical device according to claim 1, wherein the implantable medical device is configured to communicate information relating to the burden measure to an external device.

7. The implantable medical device according to claim 1, wherein the processing module is configured to identify a ventricular contraction event based on said electrocardiogram signals, compute at least one discrimination metric value for said ventricular contraction event, compare said at least one discrimination metric value to at least one of a first reference value computed based on a first number of prior ventricular contraction events and a second reference value computed based on a second number of subsequent ventricular contraction events, and classify said ventricular contraction event as a premature ventricular contraction event based on said comparison.

8. The implantable medical device according to claim 7, wherein the processing module is configured to compute said at least one discrimination metric value, with relation to a signal portion relating to said ventricular contraction event, based on a maximum positive amplitude, a maximum negative amplitude, a maximum rectified amplitude, a peak-to-peak amplitude, a maximum first derivative value, a maximum second derivative value, an area value under a curve of said signal portion until a first zero-crossing, an area value under a curve of said signal portion between a first zero-crossing and a second zero crossing, an area value under a curve of said signal portion between a second zero-crossing and a third zero crossing, a time duration value until a first zero-crossing, a time duration value between a first zero-crossing and a second zero crossing, a time duration value between a second zero-crossing and a third zero crossing and/or a time duration value between an upward crossing and a downward crossing of a ventricular detection threshold.

9. The implantable medical device according to claim 7, wherein the processing module is configured to compute said first reference value based on a first statistical measure relating to said first number of prior ventricular contraction events and/or compute said second reference value based on a second statistical measure relating to said second number of subsequent ventricular contraction events.

10. The implantable medical device according to claim 7, wherein the processing module is configured to classify said ventricular contraction event as a premature ventricular contraction event if said at least one discrimination metric value deviates from said first reference value by more than a first margin and/or deviates from said second reference value by more than a second margin.

11. The implantable medical device according to claim 7, wherein the processing module is configured to compute multiple discrimination metric values for different discrimination metrics for said ventricular contraction event and to compare the multiple discrimination metric values to multiple first reference values computed based on the first number of prior ventricular contraction events and/or multiple second reference values computed based on the second number of subsequent ventricular contraction events

12. The implantable medical device according to claim 11, wherein the processing module is configured to classify said ventricular contraction event as a premature ventricular contraction event if, based on said comparison, at least for a subset of said discrimination metric values a set of predefined conditions is fulfilled

13. The implantable medical device according to claim 7, wherein the processing module is configured to classify said ventricular contraction event as a premature ventricular contraction event based on said comparison and in addition based on a first timing distance between the ventricular contraction event and an immediately prior ventricular contraction event and/or a second timing distance between the ventricular contraction event and an immediately subsequent ventricular contraction event.

14. The implantable medical device according to claim 13, wherein the processing module is configured to evaluate, for said classifying, whether the first timing distance between the ventricular contraction event and an immediately prior ventricular contraction event is smaller than a first timing threshold and/or whether the second timing distance between the ventricular contraction event and an immediately subsequent ventricular contraction event is larger than a second timing threshold.

15. A method for operating an implantable medical device for sensing electrocardiogram signals, the method comprising:
sensing electrocardiogram signals using an arrangement of electrode poles of the implantable medical device; and
processing electrocardiogram signals obtained by the arrangement of electrode poles using a processing module of the implantable medical device;
wherein the processing module identifies, based on said electrocardiogram signals obtained by the arrangement of electrode poles, premature ventricular contraction events over a prolonged period of time and computes a burden measure indicative of the occurrence of premature ventricular contraction events in at least a portion of said prolonged period of time.
